(19) United States
(12) Patent Application Publication
(10) Pub. No.: US 2026/0235629 A1
(43) Pub. Date: Aug. 13, 2026

(54) ENDOTHELIAL AND BLOOD-BRAIN BARRIER MARKERS OF NEUROLOGICAL DISORDERS

(71) Applicant: Rawan Tarawneh, St Louis, MO (US)

(72) Inventor: Rawan Tarawneh, St Louis, MO (US)

(21) Appl. No.: 19/362,365

(22) Filed: Oct. 18, 2025

Related U.S. Application Data
(60) Provisional application No. 63/709,213, filed on Oct. 18, 2024.

(51) Int. Cl. G01N 33/68; G16H 50/30

(52) U.S. Cl. CPC G01N 33/6896; G16H 50/30; G01N 2333/47; G01N 2800/2814; G01N 2800/2821; G01N 2800/52

(57) ABSTRACT

Disclosed are methods, compositions, and computer-implemented systems for diagnosing, staging, and monitoring neurological and neurovascular disorders, including Alzheimer's disease, vascular dementia, traumatic brain and spinal-cord injury, stroke, demyelinating disease, and central nervous system vasculitis. The invention involves measuring levels of endothelial and blood-brain-barrier-associated biomarkers—comprising claudins 1-34 (including claudin-14 and claudin-23), endothelins (ET-1 to ET-3), ESAM, ESM1 (Endocan), neuropilins (NRP1 and NRP2), ZIC1, FOXP2, and neuroligins (NLGN 1-4 and subtypes)—in biological samples such as cerebrospinal fluid, plasma, serum, or other biofluids. Altered biomarker patterns indicate endothelial activation, barrier dysfunction, or neurovascular signaling imbalance associated with disease progression or therapeutic response. Also provided are analytical kits containing capture reagents, calibration standards, and a non-transitory computer-readable medium configured to integrate biomarker data, as well as machine-learning models trained to generate diagnostic, prognostic, or vascular-safety indices supporting individualized management of neurodegenerative and neurovascular conditions.

This application claims the benefit of priority under 35 U.S.C. § 119(e) to U.S. Provisional Patent Application No. 63/709,213, filed Oct. 18, 2024, entitled “Endothelial and Blood-Brain Barrier Markers of Neurological Disorders,” the entire disclosure of which is incorporated herein by reference.

### FEDERALLY SPONSORED RESEARCH AND DEVELOPMENT

This invention was made with support under National Institutes of Health-National Institute on Aging Grant RF1AG083744. The U.S. Government has certain rights in the invention.

### FIELD OF INVENTION

The invention relates to biomarkers for neurological and neurodegenerative diseases. More particularly, it concerns diagnostic, prognostic, and therapeutic-monitoring methods utilizing endothelial-associated and blood-brain-barrier (BBB)-associated proteins, including neuroligins that mediate neuronal-endothelial signaling.

### BACKGROUND OF THE INVENTION

Alzheimer's disease (AD) and related dementias lack reliable fluid biomarkers that reflect endothelial dysfunction and blood-brain barrier (BBB) integrity. Existing assays, including amyloid-β (Aβ), total tau, phosphorylated tau (p-tau), and neurofilament light chain (NfL), primarily capture neuronal injury and neurodegeneration but poorly represent vascular contributions to the disease process.

Emerging evidence demonstrates that vascular dysregulation, endothelial activation, and BBB leakage are not merely secondary events but represent core and early pathological features of AD. Endothelial dysfunction has been shown to precede and promote amyloid deposition and tau aggregation, serving as a mediator of both vascular and parenchymal injury. Disruption of endothelial junctions leads to impaired clearance of Aβ across the BBB, chronic inflammation, and metabolic stress on neuronal and glial cells. Likewise, loss of endothelial homeostasis contributes to oxidative stress, perivascular tau accumulation, and downstream synaptic failure.

Despite this growing recognition, reliable biomarkers of endothelial injury and BBB integrity remain limited, hindering efforts to quantify and therapeutically target these processes. There is a critical need for fluid-based indicators that sensitively reflect endothelial activation, tight-junction disruption, and neurovascular unit dysfunction, enabling both mechanistic insight and clinical translation.

Proteins such as claudins, ESAM, endothelins (ET-1, ET-2, and ET-3), neuropilins (NRP1 and NRP2), and ESM1 (Endocan) play essential roles in endothelial adhesion, angiogenic signaling, vasomotor regulation, and tight-junction maintenance, providing both structural and signaling control of blood-brain barrier (BBB) integrity. Endothelins are potent endothelial-derived peptides that regulate vascular tone, permeability, and inflammatory activation, while NRP1 and NRP2 act as vascular endothelial growth factor (VEGF) and semaphorin co-receptors coordinating endothelial-neuronal cross-talk, vascular remodeling, and barrier permeability regulation. Transcription factors ZIC1 and FOXP2 regulate vascular gene expression and endothelial stability, integrating transcriptional and junctional control of the BBB. Neuroligins (NLGN 1-4) bridge synaptic adhesion and endothelial communication, forming a molecular interface between neuronal and vascular compartments that transmits injury and stress signals across the neurovascular unit.

There remains a need for integrated diagnostic and monitoring tools encompassing these endothelial and BBB-related biomarkers to better understand, detect, and track endothelial contributions to AD pathogenesis and to other neurological disorders characterized by vascular dysfunction, including vascular dementia, Lewy body dementia, demyelinating disorders, stroke, neuroinflammatory, and traumatic brain or spinal cord injury.

### SUMMARY OF THE INVENTION

The present invention relates to diagnostic, prognostic, and therapeutic-monitoring methods, compositions, and computer-implemented systems for detecting and managing neurological and neurovascular diseases. The invention is based on the discovery that specific patterns of endothelial and blood-brain-barrier (BBB)-associated biomarker levels in biological fluids reflect early and progressive disruption of neurovascular integrity and signaling.

The disclosure also provides support for a method for monitoring progression of a neurological disease and/or disorder in a patient, comprising: (a) obtaining biological samples from the patient at least two or more, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and/or more, timepoints, (b) measuring levels of at least one biomarker, (c) comparing biomarker levels between timepoints, and/or (d) determining progression, stabilization, and/or improvement based on longitudinal changes in the said biomarker levels. In a first example of the method, serial increases in the levels of at least one biomarker selected from a group consisting of NRP1 and/or NRP2, endothelin, ESM1, claudin-14, claudin-23, and/or any combination thereof, together with decreased in levels of at least one biomarker selected from a group consisting of neuroligins, ESAM, FOXP2, ZIC1, and/or any combination thereof relative to baseline predict progression from preclinical to symptomatic Alzheimer's disease. In a second example of the method, optionally including the first example, longitudinal increases in levels of claudin-14, claudin-23, ESM1, NRP1, and/or endothelin correlate with worsening outcome after stroke and/or traumatic brain injury. In a third example of the method, optionally including one and/or both of the first and/or second examples, the at least one biomarker is selected from a group consisting of claudins 1-34, ZIC1, FOXP2, neuroligins 1-4 (including subtypes 1A, 1B, 1C, 2C, 2ECD, 3A, 3B, 4X, 4Y), endothelins 1-3, ESAM, ESMI, neuropilins (NRP1 and/or NRP2), and/or combination thereof. In a fourth example of the method, optionally including one and/or more and/or each of the first through third examples, the neurological disease and/or disorder patient carries an apolipoprotein E4 (APOE4) allele, and/or wherein progression in the at least one biomarker is monitored in the said patient carrying the apolipoprotein E4 (APOE4) allele. In a fifth example of the method, optionally including one and/or more and/or each of the first through fourth examples, progression is determined using a composite index computed from neuroligin and/or endothelial biomarker levels that reflects worsening neurovascular dysfunction relative to baseline. It is understood that the steps of the disclosed methods are not limited to the precise order described herein, and the steps may be performed sequentially, concurrently, or in a different order, and additional steps may be included or omitted as appropriate for a given implementation.

The disclosure also provides support for a method of monitoring effectiveness and/or safety of a therapy for a neurological disease and/or disorder in a patient, comprising: (a) obtaining a baseline biological sample from the patient before and/or at initiation of therapy, (b) measuring levels of at least one biomarker, (c) obtaining a subsequent biological sample during and/or after therapy (d) comparing biomarker levels between a baseline and/or subsequent sample, and/or (e) determining whether to continue, modify, and/or discontinue therapy based on the said comparison. In a first example of the method, biomarker monitoring is used to determine whether to reinitiate and/or permanently discontinue anti-amyloid therapy. In a second example of the method, optionally including the first example, the therapy comprises administration of an anti-amyloid monoclonal antibody, including lecanemab, donanemab, and/or a functionally equivalent disease-modifying agent for dementia, and/or wherein biomarker level changes are used to monitor risk of amyloid-related imaging abnormalities (ARIA). In a third example of the method, optionally including one and/or both of the first and/or second examples, (a) a chronic biomarker pattern characterized by decreased levels of ESAM, FOXP2, and/or ZIC1 levels together with persistently elevated ESM1, claudin-14, claudin-23, endothelin, NRP1, and/or NRP2 levels indicates increased susceptibility to amyloid-related imaging abnormalities (ARIA), and/or (b) an acute biomarker pattern characterized by further increases in ESM1, claudin-14, claudin-23, endothelin, NRP1, and/or NRP2 and/or transient elevation and/or fluctuation of ESAM, FOXP2, or ZIC1 relative to baseline indicates active and/or imminent ARIA development. In a fourth example of the method, optionally including one and/or more and/or each of the first through third examples, amyloid-related imaging abnormality (ARIA) resolution and/or recovery is indicated by (a) decreased levels of EMS1, endothelin, claudin-14, claudin-23, NRP1, and/or NRP2 relative to values observed during ARIA onset, and/or (b) stabilization and/or restoration of ESAM, FOXP2, and/or ZIC1 levels toward baseline following therapy modification and/or interruption. It is understood that the steps of the disclosed methods are not limited to the precise order described herein, and the steps may be performed sequentially, concurrently, or in a different order, and additional steps may be included or omitted as appropriate for a given implementation.

The disclosure also provides support for a kit for diagnosing and/or monitoring a neurological disease and/or disorder, comprising: (a) a panel of binding reagents selected from a group consisting of antibodies, antibody fragments, aptamers, nucleic-acid probes, and/or other binding reagents specific for at least one biomarker, and/or any combination thereof, (b) calibration standards, and/or (c) instructions for comparing biomarker levels to a control and/or reference to determine disease presence, stage, progression, and/or therapy response. In a first example of the system, the panel of binding reagents is provided within a multiplex immunoassay, microarray, nucleic-acid detection platform, and/or other analytical system configured for simultaneous quantification of the said biomarkers, the kit further configured for use in enzyme-linked immunosorbent assay (ELISA), proximity-extension assay, mass-spectrometry-based quantification, and/or equivalent analytical formats. In a second example of the system, optionally including the first example, the calibration standards comprise at least one component selected from a group consisting of pre-quantified recombinant and/or synthetic peptides, protein fragments, full-length proteins, nucleic-acid constructs, and/or any combination thereof, each corresponding to at least one biomarker selected from a group consisting of claudins 1-34 (including claudin-14 and/or claudin-23), ESAM, EMS1 (Endocan), FOXP2, ZIC1, neuroligins 1-4, neuropilins (NRP1 and/or NRP2), endothelins 1-3, and/or any combination thereof. In a third example of the system, optionally including one and/or both of the first and/or second examples, the at least one biomarker is selected from a group consisting of claudins 1-34, ZIC1, FOXP2, neuroligins 1-4 and/or subtypes 1A, 1B, 1C, 2C, 2ECD, 3A, 3B, 4X, 4Y, endothelins 1-3, ESAM, EMS1, neuropilins (NRP1 and/or NRP2), and/or any combination thereof, and/or wherein the kit comprises capture reagents for at least one neuroligin and/or at least one endothelial biomarker selected from a group consisting of ESAM, ESM1, claudin-14, claudin-23, an endothelin, a neuropilin (NRP1 or NRP2), and/or any combination thereof. It is understood that the steps of the disclosed methods are not limited to the precise order described herein, and the steps may be performed sequentially, concurrently, or in a different order, and additional steps may be included or omitted as appropriate for a given implementation.

The disclosure also provides support for a computer-implemented method for generating a disease risk or stage score in a patient, comprising: (a) receiving biomarker level data measured from a biological sample, (b) normalizing at least one biomarker level against a reference database or baseline, (c) applying a classifier trained to distinguish between neurological disease states and control subjects, and (d) outputting a diagnostic or prognostic score indicating a likelihood or stage of disease, wherein the classifier is configured to assign feature weights comprising (a) increasing a computed probability of early alzheimer's disease for patterns characterized by elevated levels of neuroligins, ESM1, endothelin, claudin-14, claudin-23, NRP1, and NRP2, together with reduced levels of ESaM, FOXP2, and ZIC1, and (b) increase the computed probability of advanced alzheimer's disease for patterns characterized by reduced levels of neuroligins, FOXP2, and ZIC1. In a first example of the method, the method further comprises: a non-transitory computer-readable medium storing software executable instructions configured to (a) receiving the at least one biomarker-level data from at least one assay, (b) normalizing levels of at least one biomarker relative to at least one reference datasets, and (c) generating diagnostic, prognostic, and therapeutic-response indices, and wherein the further including stage-specific classification of early (neuroligin increase) versus late (neuroligin decreased) disease. In a second example of the method, optionally including the first example, the method further comprises: integration of said machine-learning or artificial-intelligence model with a clinical-decision support platform configured for real-time disease stratification, risk prediction, or therapy-response optimization. It is understood that the steps of the disclosed methods are not limited to the precise order described herein, and the steps may be performed sequentially, concurrently, or in a different order, and additional steps may be included or omitted as appropriate for a given implementation.

In certain embodiments, the invention provides methods for measuring levels of one or more biomarkers selected from claudins (including claudin-14 and claudin-23), ESAM, EMS1 (Endocan), endothelins (ET-1, ET-2, ET-3), neuropilins (NRP1 and NRP2), transcriptional regulators FOXP2 and ZIC1, and neuroligins (NLGN1-4 and subtypes) in cerebrospinal fluid, plasma, serum, or other biological samples.

Alterations in these biomarkers, individually or in combination, provide sensitive indicators of BBB permeability, endothelial activation, transcriptional dysregulation, and neurovascular coupling abnormalities that occur in Alzheimer's disease and other neurological conditions.

The invention further provides stage-specific diagnostic and prognostic algorithms that distinguish early or preclinical disease-characterized by elevations in neuroligins, ESM1, NRP1/NRP2, and endothelins with decreased ESAM-from later stages marked by declines in neuroligins, FOXP2, and ZIC1 and persistent endothelial injury. In some embodiments, the biomarkers are used longitudinally to monitor progression, treatment response, or vascular safety during disease-modifying therapy, including anti-amyloid monoclonal-antibody treatment.

In additional embodiments, the invention provides kits comprising capture reagents, calibration standards, and computer-readable software for quantifying and interpreting biomarker data.

The computer-implemented systems employ machine-learning classifiers or statistical models to normalize biomarker inputs, generate diagnostic or prognostic scores, and output individualized disease-stage or vascular-risk assessments.

Applications of the invention extend beyond Alzheimer's disease to include vascular dementia, frontotemporal dementia, Lewy body dementia, traumatic brain and spinal-cord injury, stroke, autoimmune and demyelinating disorders (including multiple sclerosis, ADEM, NMOSD, and MOGAD), neuroinflammatory conditions including central nervous system vasculitis, and neuroinfectious conditions.

Across these indications, the disclosed biomarkers serve as accessible, fluid-based indicators of endothelial integrity and BBB function, enabling early detection, risk stratification, therapeutic monitoring, and precision management of neurovascular disorders.

### BRIEF DESCRIPTION OF DRAWINGS

FIG. 1 shows correlations between cerebrospinal fluid (CSF) NLGN1A levels and established markers of AD pathology.

FIG. 2 shows differences in CSF EMS1 and ESAM levels between cognitively normal controls and cognitively impaired individuals.

FIG. 3. Baseline cerebrospinal-fluid NRP1 levels predict longitudinal cognitive decline in preclinical Alzheimer's disease.

FIG. 4 shows the correlation between CSF claudin-23 levels and Preclinical Alzheimer's Composite (PACC) scores (global cognition).

FIG. 5 A. Endothelial-enriched expression of claudin-14 (CLDN14) across human brain vascular cell types.

FIG. 5B. Endothelial-restricted expression of claudin-5 (CLDN5) across brain arteriolar, capillary, and venular endothelial populations.

FIG. 5C. Selective expression of endothelial cell-selective adhesion molecule (ESAM) in brain vascular endothelial compartments.

### DETAILED DESCRIPTION OF THE INVENTION

Examples of embodiments are provided so that this disclosure will be thorough and fully convey the scope to those skilled in the art. Numerous specific details are set forth, such as examples of specific components, devices, and methods, to provide a thorough understanding of the embodiments of the present disclosure. It will be apparent to those skilled in the art that specific details need not be employed, that example embodiments may be embodied in many different forms, and that neither should be construed to limit the scope of the disclosure. In some examples, embodiments, aspects, well-known processes, well-known device structures, and well-known technologies are not described in detail least one specification heading is required.

The terminology used herein is for the purpose of describing particular example embodiments only and is not intended to be limiting. As used herein, the singular forms “a,” “an,” and “the” may be intended to include the plural forms as well unless the context clearly indicates otherwise. The terms “comprises,” “comprising,” “including,” and “having,” are inclusive and therefore specify the presence of stated features, integers, steps, operations, elements, and/or components but do not preclude the presence or addition of one or more other features, integers, steps, operations, elements, components, and/or groups thereof. The method steps, processes, and operations described herein are not to be construed as necessarily requiring their performance in the particular order discussed or illustrated unless specifically identified as an order of performance. It is also to be understood that additional or alternative steps may be employed.

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. When a definition is provided herein, it supersedes any other meaning or definition.

Where a range of values is provided, it is to be understood that every intervening value, to at least the tenth of the unit of the lower limit unless the context clearly indicates otherwise, between the upper and lower limits of that range, as well as any other stated or intervening value within that range, is encompassed by the invention. The upper and lower limits of any such range may be independently included or excluded, as indicated by the context, and all sub-ranges and combinations of sub-ranges between the stated limits are also encompassed by the invention unless specifically excluded. In embodiments where a biomarker or disease is specified as a member of one or more Markush groups, only those biomarkers or diseases that are biologically relevant, measurable, and capable of demonstrating the described diagnostic or prognostic association are intended to be included.

It is understood that the steps of the disclosed methods are not limited to the precise order described herein, and may be performed in a different sequence, simultaneously, or with intervening steps as appropriate for a particular implementation. Additional steps may be added, and one or more of the recited steps may be omitted, combined, or subdivided without departing from the spirit and scope of the invention. Variations in step order or inclusion of supplementary steps that achieve substantially the same diagnostic, prognostic, or monitoring purpose are considered within the scope of the disclosed methods.

Unless defined otherwise, all technical and scientific terms used herein have the same meaning as commonly understood by one of ordinary skill in the art to which the present invention pertains. Although any methods and materials substantially similar or equivalent to those described herein can also be used in the practice or testing of the invention, the preferred and/or optional methods and materials are described below.

It is further to be noted that, as used herein and in the appended claims, the singular forms “a,” “an,” and “the” include their plural referents unless the context clearly dictates otherwise.

Definitions: For clarity and to avoid ambiguity, the following terms used in this disclosure are defined as follows:
“Biological sample” encompasses any specimen obtained from a subject suitable for biomarker measurement, including but not limited to cerebrospinal fluid (CSF), plasma, serum, blood, urine, saliva, or tissue lysates, as well as derivatives or fractions thereof (for example, exosomes, cell-free supernatants, or isolated protein fractions).
“Endothelial biomarker” refers to any molecule that is predominantly expressed, released, and/or shed by vascular endothelial cells and participates in maintaining, signaling, and/or regulating BBB integrity, permeability, angiogenesis, and/or vascular homeostasis.
“Neuroligin” refers to any isoform, fragment, and/or subtype of neuroligin proteins (NLGN1-NLGN4), including but not limited to subtypes 1A, 1B, 1C, 2C, 2ECD, 3A, 3B, 4X, and 4Y, and/or biologically active variants, post-translationally modified forms, and/or soluble ectodomains thereof.
“Neuropilin” refers to neuropilin-1 (NRP1) and/or neuropilin-2 (NRP2), including full-length, splice variants, post-translationally modified forms, shed/soluble ectodomains, and/or fragments thereof that retain biomarker utility.
“Diagnosis” means determining and/or classifying the likelihood, presence, stage, and/or subtype of a neurological or neurovascular disease and/or disorder in a subject based on quantitative or qualitative comparison of one or more biomarker levels to a control, reference population, and/or baseline measurement.
“Classifier” refers to a computer-implemented and/or algorithmic system configured to receive biomarker data (for example, protein levels and/or normalized assay values) and to compute, output, and/or display a diagnostic, prognostic, and/or therapeutic-response score, probability, and/or index indicative of disease status or trajectory.

### Biomarkers and Pathophysiologic Rationale

Claudins 1-34 (including claudin-14 and claudin-23): Claudins form the structural backbone of tight junctions in endothelial barriers, including the blood-brain barrier (BBB). Members of this family, particularly claudin-14 and claudin-23, are thought to regulate paracellular permeability, ion selectivity, and tight-junction cohesion in brain microvascular endothelium. In some embodiments, disruption or remodeling of claudin organization is indicative of early compromise of barrier integrity, potentially leading to increased vascular permeability and parenchymal exposure to circulating factors. Claudins may undergo proteolytic cleavage or vesicular release, generating soluble fragments detectable in cerebrospinal fluid (CSF), plasma, or serum. These shed forms can act as indicators of tight-junction remodeling and endothelial stress. In certain embodiments, altered levels-either elevations suggesting junctional activation or reductions suggesting diminished synthesis or chronic endothelial loss-are interpreted as evidence of BBB perturbation. When evaluated with complementary biomarkers such as ESAM, EMS1, FOXP2, ZIC1, endothelin, NRP1/NRP2, or neuroligins, claudin measurements enhance the sensitivity and specificity of diagnostic panels for detecting early BBB dysfunction and neurovascular injury across neurological disorders.

ESAM (endothelial cell-selective adhesion molecule): ESAM is a type I transmembrane adhesion protein localized to endothelial junctions, where it contributes to vascular stability and regulated solute exchange. In certain embodiments, proteolytic processing of ESAM by metalloproteases produces soluble forms measurable in biofluids. Variations in ESAM levels can therefore serve as indicators of junctional disassembly, vascular inflammation, or altered permeability. Monitoring ESAM in CSF or plasma may provide a means of assessing BBB integrity and endothelial remodeling. When analyzed in conjunction with claudins, EMS1, endothelin, FOXP2, ZIC1, NRP1/NRP2, or neuroligins, ESAM contributes to a multidimensional index of endothelial function that supports diagnosis and staging of neurodegenerative and vascular disorders.

EMS1 (Endocan): EMS1 is a soluble dermatan-sulfate proteoglycan secreted by endothelial cells under pro-angiogenic or inflammatory conditions. It modulates permeability and leukocyte adhesion and is measurable in plasma and CSF. In various embodiments, altered EMS1 levels may represent endothelial activation, microvascular stress, or BBB dysregulation. Because EMS1 is secreted rather than only shed, it can serve as a robust marker of endothelial response dynamics. Temporal assessment of ESM1 provides information regarding disease progression, therapeutic response, or vascular effects of pharmacologic intervention, including anti-amyloid therapies. In combined panels incorporating ESAM, endothelin, claudins, FOXP2, ZIC1, NRP1/NRP2, and neuroligins, EMS1 enables differentiation of inflammatory versus degenerative vascular states and supports individualized monitoring of neurovascular health.

FOXP2 and ZIC1: FOXP2 and ZIC1 are transcription factors implicated in neuronal differentiation, vascular development, and maintenance of BBB integrity. Both exhibit endothelial expression and regulate networks controlling angiogenesis, junctional stability, and neuronal-endothelial communication. In some embodiments, reduced or altered activity of FOXP2 and ZIC1 is associated with dysregulation of vascular gene expression and loss of endothelial homeostasis. Their coordinated variation with neuroligins may define a transcriptional signature of neurovascular stress. When incorporated into biomarker panels, FOXP2 and ZIC1 enhance the mechanistic interpretability of fluid assays by linking observed molecular changes to endothelial transcriptional pathways.

Neuropilins (NRP1 and NRP2): NRP1 and NRP2 are transmembrane co-receptors for vascular endothelial growth factor (VEGF) and class-3 semaphorins that mediate neurovascular communication, angiogenic signaling, and barrier maintenance. The inventor has identified alterations in NRP1 and NRP2 levels in cerebrospinal fluid associated with neurodegenerative progression and endothelial stress. In various embodiments, elevated NRP1/NRP2 levels serve as indicators of endothelial activation or remodeling within the BBB, while normalization may correspond to vascular recovery. Serial quantification of NRP1/NRP2 can therefore provide a pharmacodynamic indicator of endothelial or synaptic repair. In multiplex panels, coordinated changes in NRP1/NRP2 together with EMS1 or claudins, and reciprocal changes in ESAM or FOXP2, delineate molecular phenotypes of early neurovascular dysfunction.

Neuroligins: Dynamic Indicators of Neurovascular and Synaptic Stress: Neuroligins (NLGN 1-4 and subtypes) are postsynaptic adhesion molecules that also influence endothelial signaling and neurovascular coupling. They regulate synaptic stability and participate in cross-talk among neurons, astrocytes, and endothelial cells. In certain embodiments, neuroligin levels exhibit stage-dependent trajectories, wherein relative elevation in preclinical or early disease may reflect compensatory up-regulation, and reduction in symptomatic stages may reflect synaptic and endothelial decompensation. Soluble neuroligin fragments detectable in CSF or plasma thus provide stage-specific biochemical signatures of synaptic-vascular stress. When analyzed with endothelial markers such as ESAM, EMS1, claudin-14/23, NRP1/NRP2, and transcriptional factors FOXP2 and ZIC1, neuroligins augment both the sensitivity and staging precision of the diagnostic platform.

Endothelins (ET-1, ET-2, ET-3): Endothelins are small vasoactive peptides primarily produced by endothelial cells that regulate vascular tone, permeability, and paracrine signaling within the neurovascular unit. Dysregulated endothelin activity is believed to contribute to impaired perfusion and barrier instability in neurodegenerative states. In certain embodiments, alterations in ET-1 or ET-2 levels may correspond to endothelial activation or oxidative stress, whereas ET-3 variation may indicate compensatory vasodilatory adaptation. Measurement of endothelins in combination with ESAM, EMS1, NRP1/NRP2, claudins, FOXP2, ZIC1, and neuroligins provides an integrated assessment of microvascular function and enables early detection of endothelial perturbation preceding overt neurodegeneration.

### Diagnostic Methods

A diagnostic method comprises obtaining a biological sample from a subject, measuring the level of one or more (at least one) biomarkers selected from claudins 1-34 (including claudin-14 and claudin-23), ESAM, EMS1 (Endocan), endothelins (ET-1, ET-2, ET-3), ZIC1, FOXP2, neuropilins (NRP1 and NRP2), and/or neuroligins (NLGN 1-4 and subtypes 1A, 1B, 1C, 2C, 2ECD, 3A, 3B, 4X, 4Y); comparing the biomarker levels to a control or reference standard; and identifying a neurological and/or neurovascular disorder when one and/or more biomarker levels deviate from the reference pattern.

The biological sample may comprise cerebrospinal fluid (CSF), plasma, serum, urine, or saliva, and/or any other biofluid and/or tissue specimen suitable for molecular analysis.

Altered levels of one or more biomarkers reflect underlying endothelial injury, BBB disruption, vascular inflammation, or impaired neurovascular communication. In certain embodiments, decreased ESAM or FOXP2, increased NRP1/NRP2, elevated ESM1, endothelins, and/or claudin-23, and/or stage-dependent alterations in neuroligins indicate endothelial activation or BBB compromise associated with neurological pathology.

In illustrative examples, such biomarker changes may distinguish Alzheimer's disease (AD), vascular dementia, and/or Lewy body dementia from cognitively normal states; identify vascular involvement in stroke, traumatic brain injury, and/or demyelinating disorders; and/or indicate early neurovascular dysfunction preceding overt neurodegeneration.

### Monitoring Progression and Therapeutic Response

Serial and/or longitudinal measurements of one or more biomarkers at two or more (alt east two) timepoints are used to assess disease trajectory, treatment response, or recovery.

Changes in relative levels of endothelial-and BBB-associated biomarkers, such as claudins (including claudin-14 and claudin-23), ESAM, EMS1, endothelins, NRP1/NRP2, ZIC1, FOXP2, and/or neuroligins, provide dynamic indicators of neurovascular status.

A decline in neuroligin levels in conjunction with increases in NRP1/NRP2, endothelins, and/or EMS1 and/or decreases in ESAM may indicate progression from preclinical to symptomatic neurodegenerative disease, reflecting worsening BBB dysfunction and/or impaired neurovascular signaling. Conversely, stabilization and/or normalization of these biomarker ratios may indicate disease stabilization or therapeutic benefit.

During anti-amyloid therapy (for example, lecanemab or donanemab), rising levels of claudin-14, claudin-23, NRP1/NRP2, endothelins, and/or EMS1 may signal emerging endothelial stress and/or risk of amyloid-related imaging abnormalities (ARIA), while subsequent reduction or normalization of these markers following treatment modification is indicative of recovery or improved vascular integrity.

These biomarker trends can thus be used to guide treatment decisions, evaluate therapeutic safety, and/or monitor vascular contributions to disease progression across a range of neurological and neurovascular disorders.

### Potential Applications

The disclosed endothelial-and blood-brain-barrier (BBB)-associated biomarkers have diagnostic, prognostic, and/or therapeutic-monitoring utility across a spectrum of neurological and/or neurovascular disorders. In various embodiments, biomarker panels comprising claudins (particularly claudin-14 and claudin-23), endothelins (ET-1 to ET-3), ESAM, EMS1 (Endocan), neuropilins (NRP1 and NRP2), ZIC1, FOXP2, and/or neuroligins (NLGN 1-4 and subtypes) are used to evaluate endothelial activation, junctional remodeling, transcriptional regulation, vasomotor signaling, and/or neurovascular coupling.

Alterations in one or more (at least one) of these biomarkers may indicate changes in BBB integrity and/or endothelial function that precede and/or accompany neuronal injury.

### 1. Alzheimer's Disease and Related Neurodegenerative Disorders

In certain embodiments, biomarker combinations are utilized for early detection, differential diagnosis, prognosis, and therapy monitoring in Alzheimer's disease (AD) and/or related dementias.

Early detection: Relative elevations in NRP1/NRP2, EMS1, endothelins, and/or claudin-23, together with lower levels of ESAM, FOXP2, and/or ZIC1, are interpreted as indicative of early endothelial activation and/or vasoconstrictive stress, potentially preceding tau accumulation and/or measurable cognitive decline.

Differential diagnosis: Composite indices such as (NRP1+ESM)/claudin-23, optionally incorporating ZIC1, ESAM1, and/or endothelins, may differentiate AD-type endothelial signatures from those observed in other dementias by quantifying distinct endothelial-transcriptional relationships.

Prognosis: Progressive increases in NRP1/NRP2, ESM1, endothelins, and claudin-23, followed by reductions in neuroligins, FOXP2, and/or ZIC1, can be modeled as indicative of transition from preclinical to symptomatic disease, reflecting evolving neurovascular dysregulation.

Therapeutic monitoring: Serial assessment of NRP1/NRP2, claudin-14, claudin-23, endothelins, and/or ESM during anti-amyloid and/or anti-tau therapy provides information on vascular safety and/or treatment response, detecting early BBB stress before clinical events.

### 2. Prediction and Monitoring of Amyloid-Related Imaging Abnormalities (ARIA)

In certain embodiments, patterns involving higher EMS1, endothelins, and/or claudins-14/-23 combined with or without lower ESAM, FOXP2, and/or ZIC1 are used as candidate indicators of vascular stress potentially associated with amyloid-related imaging abnormalities (ARIA) during monoclonal-antibody treatment.

These biomarkers may be integrated into predictive algorithms and/or clinical-decision-support systems configured to flag individuals displaying early endothelial and/or BBB strain, thereby supporting imaging follow-up or therapy adjustment. This embodiment provides a framework for personalized vascular-safety monitoring and proactive management of therapeutics with neurovascular effects.

### 3. Traumatic Brain and Spinal-Cord Injury

In some embodiments, acute variations in claudin-14, claudin-23, NRP1, NRP2, EMS1, ESAM, ZIC1, and/or endothelins are evaluated as indicators of BBB disruption, endothelial activation, and/or vasomotor imbalance following traumatic injury. Magnitude and/or persistence of these changes can be analyzed to estimate injury severity and/or monitor response to neurocritical-care interventions such as osmotic therapy, hypothermia, or decompressive procedures.

### 4. Stroke and Other Neurovascular Disorders

In ischemic and/or hemorrhagic stroke, altered claudin-14, claudin-23, NRP1/NRP2, ESM1, ESAM, ZIC1, and/or endothelin levels may reflect vascular permeability shifts, endothelial stress, or perfusion irregularities.

Sequential biomarker assessment can be used to evaluate infarct progression, hemorrhagic transformation, and/or reperfusion outcomes. In certain embodiments, endothelin trends are used as indices of microvascular constriction or reperfusion injury risk, providing dynamic insights into vascular tone regulation after stroke.

### 5. Demyelinating and Neuroinflammatory Disorders

In autoimmune and/or inflammatory demyelinating conditions-including multiple sclerosis (MS), acute disseminated encephalomyelitis (ADEM), neuromyelitis optica spectrum disorder (NMOSD), and/or MOG-antibody disease (MOGAD) the disclosed biomarkers are used to monitor endothelial and/or BBB involvement.

Active disease: Increased claudin-14, claudin-23, NRP1/NRP2, ESM1, ESAM, and endothelins are interpreted as consistent with acute endothelial inflammation, lesion activity, and/or vasospasm.

Treatment response: Normalization and/or reduction of these biomarkers may indicate restoration of endothelial integrity and/or effective immunomodulation.

Transcriptional dysregulation: Reduced FOXP2 and/or ZIC1 levels may serve as indicators of impaired endothelial transcriptional stability and/or are used to identify patients at risk for aggressive and/or relapsing disease trajectories.

### 6. Central Nervous System Vasculitis and Neuroinfectious Disorders

In certain embodiments, simultaneous elevation of EMS1, endothelins, claudins, and NRP1/NRP2, combined with lower ESAM or FOXP2 levels, is evaluated as a pattern suggestive of diffuse endothelial activation and/or vessel-wall stress. These biomarker relationships can aid in differentiating inflammatory vasculopathies from ischemic and/or degenerative conditions and/or in monitoring responses to corticosteroid or immunosuppressive therapies.

### 7. Cross-Condition Utility and Clinical Decision Support

Across multiple neurological conditions, trajectories of endothelial and/or BBB biomarkers provide quantitative metrics for:
Risk stratification—identifying individuals with elevated likelihood of rapid progression and/or treatment-related vascular events;
Therapeutic guidance—adjusting pharmacologic and/or immunologic interventions according to vascular-stress signatures; and
Safety surveillance—detecting emerging endothelial and/or inflammatory complications that warrant early intervention.

In certain embodiments, biomarker data are processed through a computer-implemented analytic platform configured to generate individualized stage, risk, and/or treatment-response indices, supporting precision-medicine applications.

### 8. Future Extensions

The same biomarker framework can be applied to additional conditions characterized by endothelial stress and/or BBB compromise, including chronic traumatic encephalopathy (CTE), long-COVID cognitive dysfunction, viral and/or autoimmune encephalitis, pediatric leukodystrophies, and/or systemic diseases with cerebral vasculopathy (e.g., lupus, antiphospholipid syndrome, diabetes, or hypertension).

In these embodiments, analysis of endothelins, NRP1/NRP2, claudins, ESAM, ESM1, FOXP2, ZIC1, and/or neuroligins provides fluid-based indices of BBB integrity and endothelial health, enabling early detection, individualized risk assessment, and/or therapeutic monitoring across diverse neurovascular disorders.

### Kits

Diagnostic or monitoring kits comprise a panel of capture reagents-such as antibodies, antibody fragments, aptamers, nucleic-acid probes, and/or other binding molecules-specific for one or more (at least one) of the biomarkers disclosed herein, together with or without calibration standards, buffers, and/or instructions for quantitative and/or qualitative comparison of biomarker levels to reference or control values. In certain embodiments, the kit includes quality-control materials and/or reference calibrators and/or is configured for immunoassay, aptamer-based, bead-based, and/or mass-spectrometric platforms. In some embodiments, the kit additionally comprises a computer-readable medium and/or software interface configured to receive biomarker data, normalize values relative to reference datasets, and/or generate a diagnostic, prognostic, or therapeutic-response index.

### Computer-Implemented Embodiments

In certain embodiments, the system analyzes biomarker data and generates diagnostic, prognostic, and/or therapeutic-response outputs. The system receives quantitative biomarker levels (e.g., immunoassay, aptamer-based, mass-spectrometric), optionally normalizes values to calibrators, and/or applies a classifier (e.g., logistic regression, random forest, SVM, gradient-boosted trees, neural networks). In some implementations, panels include combinations of claudins, ESAM, ESM1, FOXP2, ZIC1, endothelins, NRPs, and/or neuroligins, optionally with clinical covariates. Patterns indicative of endothelial dysfunction, BBB disruption, and/or neurovascular dysregulation increase the likelihood of disease as computed. Reports display indices, trajectories, and/or risk flags; the system can run locally or via secure cloud.

### EXAMPLES

### Example 1—Neuroligin Correlations and Stage-Dependent Dynamics in Alzheimer's Disease

Comprehensive correlation analyses were conducted across multiple neuroligin isoforms (NLGN1A-C, NLGN2C, NLGN3A-B, NLGN4X-Y) in cerebrospinal fluid (CSF) from participants spanning the Alzheimer's disease (AD) continuum-from cognitively normal controls and preclinical biomarker-positive individuals to subjects with mild cognitive impairment (MCI) due to AD and AD dementia (n=923 participants).

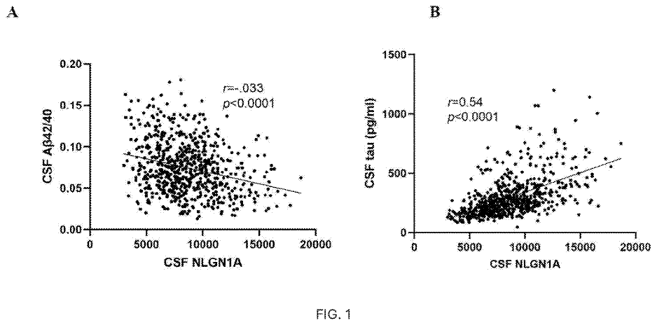

Overall correlation patterns: All major neuroligin isoforms demonstrated robust positive correlations with canonical AD biomarkers, including total tau and phosphorylated tau (p-tau181) while showing negative correlations with the Aβ42/40 ratio. These relationships with CSF tau and p-tau181 were strongest for the NLGN1 subfamily (r=0.5-0.7, p<0.0001), and with CSF Aβ42/40 (r=−0.3to −0.4, p<0.0001) and persisted across independent analytical platforms, indicating that neuroligin levels rise in parallel with tau pathology and amyloid accumulation (FIG. 1).

Stage-specific trajectories: When stratified by clinical stage, neuroligin levels followed a biphasic pattern across the AD continuum.

In preclinical or early mild cognitive impairment (MCI) stages, NLGN1-4 isoforms were elevated relative to age-matched controls, consistent with compensatory up-regulation during early synaptic and endothelial stress.

In late MCI or AD dementia, neuroligin concentrations declined significantly, reflecting synaptic exhaustion, neuronal loss, and endothelial decompensation. This trajectory parallels patterns observed for other neuroinflammatory and neurovascular response proteins such as YKL-40 and sTREM2, underscoring the role of neuroligins as dynamic indicators of evolving synaptic-vascular pathology.

Synaptic and axonal injury markers: Neuroligins were strongly correlated with established CSF markers of synaptic loss and axonal degeneration, including neurogranin, SNAP-25, VILIP-1, and neurofilament light (NfL) (r=0.5-0.7, p <0.0001). In early disease, neuroligin elevations paralleled increases in these markers, whereas in advanced stages, neuroligin declines tracked ongoing synaptic attrition. These findings support neuroligins as bidirectional indicators of synaptic-vascular interface remodeling.

Inflammatory and endothelial associations: Across stages, neuroligins exhibited significant positive correlations with inflammatory and endothelial proteins such as STREM2, YKL-40, and NRP1/NRP2 (r=0.4-0.6, all p<0.0001), consistent with coordinated activation of neuroinflammatory and neurovascular signaling networks. Elevated neuroligin levels in early disease coincided with increased endothelial activation, while subsequent neuroligin decline reflected transition to irreversible vascular and neuronal injury.

Intra-family co-regulation: Pairwise analyses demonstrated exceptionally high intra-family correlations among neuroligin isoforms (e.g., NLGN1A-1B r=0.97; NLGN1A-1C r=0.98; NLGN1-NLGN3A r=0.95; NLGN1-NLGN4X r=0.92; all p<0.0001), indicating coordinated regulation across splice variants and supporting the presence of a shared neuroligin signaling axis detectable in CSF.

Isoform-specific observations: The NLGN1 subfamily displayed the most consistent and strongest associations with tau-related pathology, with NLGN1A and NLGN1B correlating most strongly with global tau burden on tau PET (r≈0.38-0.40, p<0.0001), followed by NLGN1C, NLGN2ECD, and NLGN4X (r≈0.35-0.38). NLGN3 and NLGN4Y exhibited weaker but directionally similar trends (r≈0.2-0.3, p<0.05). Early-stage up-regulation was most pronounced for NLGN1A/B, while later-stage decline was most evident for NLGN2C and NLGN4X/Y, suggesting functional specialization in synaptic-endothelial signaling.

Interpretation: Together, these findings demonstrate that CSF neuroligin levels covary dynamically with amyloid, tau, synaptic, inflammatory, and endothelial injury markers in a stage-dependent manner. Early elevation of neuroligins reflects compensatory neurovascular activation, whereas later decline signifies structural and functional failure of the neurovascular unit. The strong intra-family co-regulation and consistent associations with tau and vascular markers position neuroligins—particularly the NLGN1 isoforms—as quantitative, stage-sensitive biomarkers for detecting and tracking synaptic-vascular deterioration across the AD spectrum.

### Example 2—Endothelial Biomarker Correlations with AD and Neurovascular Injury

Comprehensive analyses were performed across endothelial-and blood-brain-barrier (BBB)-associated biomarkers—ESAM, EMS1 (Endocan), claudin-14, claudin-23, neuropilin-1 (NRP1), FOXP2, and ZIC1—in human cerebrospinal-fluid (CSF) samples spanning the Alzheimer's disease (AD) continuum (n=923). These data were compared with amyloid and tau biomarkers, synaptic and axonal injury markers, and inflammatory proteins to delineate the vascular contribution to neurodegeneration.

Overall correlation patterns: All endothelial markers demonstrated significant associations with canonical AD biomarkers, particularly tau and p-tau181.

NRP1 and EMS1 showed strong positive correlations with total tau (r≈0.50-0.52, p<10⁻⁴) and p-tau181 (r≈0.52, p<10⁻⁴) reflective of tau pathology and neurodegeneration. NRP1 was also negatively correlated with the Aβ42/40 ratio (r≈−0.22, p<10⁻⁴), indicating linkage with higher brain amyloid burden.

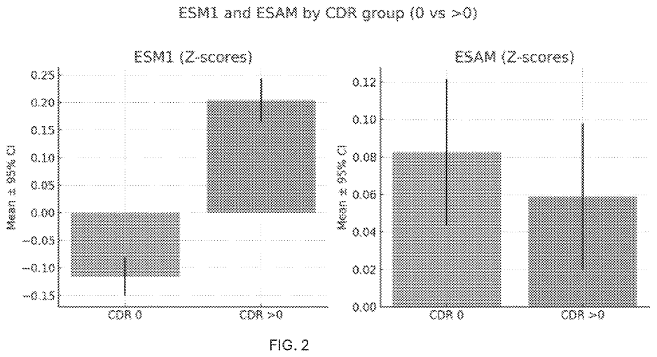

EMS1 was markedly elevated in cognitively impaired individuals (CDR>0) with AD relative to cognitively normal controls (p≈2.6×10⁻¹²) (FIG. 2).

ESAM displayed a reciprocal pattern, with significantly lower CSF levels in cognitively impaired compared to cognitively normal participants (p=0.007) (FIG. 2) and negative correlations with tau and p-tau (r≈−0.30to −0.55 ).

FOXP2 and ZIC1 exhibited inverse correlations with tau and p-tau181 (r≈−0.30 to −0.45; p=0.01), consistent with transcriptional down-regulation during progressive endothelial stress.

Synaptic and axonal injury markers: NRP1, ESM1, claudin-23, and claudin-14 correlated positively with synaptic and axonal injury proteins including neurogranin, SNAP-25, VILIP-1, and NfL (r≈0.33-0.65, all p<10⁻⁴). These patterns mirror vascular-synaptic coupling in endothelial activation. Conversely, ESAM, FOXP2, and ZIC1 were inversely correlated with these same markers (r≈−0.35 to −0.50), underscoring their roles in maintaining endothelial integrity and transcriptional stability (p<0.001).

Inflammatory and glial markers: EMS1 displayed strong positive correlations with inflammatory and glial proteins YKL-40 (r≈0.55-0.70, p<10⁻⁴) and sTREM2 (r≈0.45-0.55, p<10⁻⁴), reflecting endothelial activation and microglial co-activation. NRP1 tracked this inflammatory load (r≈0.45-0.60 vs STREM2 and YKL-40, p<10⁻⁴), aligning with its function as a vascular and inflammatory coreceptor.

Claudin-23 and claudin-14 also correlated positively with YKL-40 (r≈0.40-0.55), whereas FOXP2 and ZIC1 correlated negatively (r≈-0.30 to-0.45), indicating loss of endothelial homeostatic transcriptional control in high-inflammation states (p<0.001).

Inter-marker relationships: Within the endothelial panel, ESAM levels were inversely correlated with ESM1, NRP1, and claudin-23 (r≈−0.45 to −0.60), reflecting counter-regulation between barrier-stabilizing and barrier-disruptive pathways. FOXP2 and ZIC1 correlated strongly with each other (r≈0.75) and positively with ESAM (r≈0.50-0.65), defining a shared vascular-integrity signature (p<0.001).

Associations with amyloid and tau imaging: CSF NRP1, EMS1, claudin-23, and claudin-14 showed positive correlations with amyloid positron emission tomography (PET) centiloid values and tau PET SUVRs (r≈0.35-0.60, p<10⁻⁴). By contrast, ESAM, FOXP2, and ZIC1 demonstrated negative correlations (r≈−0.30 to −0.50), linking reduced endothelial stability with higher in-vivo amyloid and tau burden (p<0.001).

Longitudinal and clinical associations: Longitudinal analyses showed that rising NRP1, ESM1, and claudin-23 levels predicted conversion from preclinical to symptomatic AD, while concurrent decreases in ESAM, FOXP2, and ZIC1 signaled progressive cognitive decline and increasing vascular permeability (p<0.0001). These dynamic trajectories paralleled stage-dependent neuroligin changes, wherein early neuroligin elevations corresponded to endothelial activation and later declines to neurovascular failure, supporting a unified endothelial-synaptic deterioration axis.

Interpretation: Collectively, these data establish that NRP1, EMS1, and claudins 14 and 23 act as positive indicators of vascular stress and BBB leakage, whereas ESAM, FOXP2, and ZIC1 reflect protective endothelial and transcriptional integrity. Composite indices incorporating these markers-such as ESAM/NRP1 or (FOXP2+ZIC1)/(NRP1+claudin-23)-quantify endothelial health and predict disease trajectory. This integrated biomarker framework captures the transition from compensatory endothelial activation to structural vascular collapse, revealing the central role of BBB dysfunction in Alzheimer's disease pathogenesis

### Example 3—Integrated Biomarker Classifier and Predictive Modeling

To evaluate the combined diagnostic and prognostic performance of the disclosed biomarkers, a multivariate classifier was developed using quantitative CSF data from 640 participants spanning cognitively normal, mild cognitive impairment, and AD dementia groups.

Feature set. Input features included normalized concentrations of:
Endothelial markers: ESAM, EMS1 (Endocan), claudin-14, claudin-23, NRP1
Transcriptional regulators: FOXP2, ZIC1
Neuronal-endothelial markers: neuroligins (NLGN1A-C, NLGN2C, NLGN3A, NLGN4X-Y)
   together with standard CSF reference markers (Aβ42/40, tau, p-tau181) and demographic covariates (age, sex, APOE ε4 status).

Model development. The dataset was randomly divided into training (70%) and test (30%) cohorts with five-fold cross-validation. Multiple algorithms—logistic regression, random-forest ensemble, gradient-boosted trees, and multilayer neural networks—were trained and compared. Features were z-scored, and hyperparameters optimized to maximize AUC.

Model performance: Across methods, models incorporating neuroligins plus NRP1 and other endothelial biomarkers outperformed those using core AD markers alone. A combined neuroligin+endothelial+transcriptional model achieved an AUC of ≈0.93 for distinguishing AD from controls (vs. ≈0.86 using tau/p-tau/Aβ alone). Predictive accuracy for conversion from MCI to dementia reached ≈0.88±0.03(p<0.05 ). Ablation of either the neuroligin subset or the endothelial subset (including NRP1) reduced AUCs by ≈0.05-0.08, confirming complementary information content.

Feature interpretation. Feature-importance analyses showed that elevated NRP1, ESM1, claudin-14/23, and neuroligin-1 family levels, together with decreased ESAM, FOXP2, and ZIC1, contributed most strongly to higher disease-probability scores—reflecting co-occurrence of endothelial activation, BBB leakage, and synaptic-vascular dysregulation.

Predictive validation and longitudinal application. When applied to longitudinal data, the classifier predicted clinical progression over 24 months (c-index≈0.85). Participants with higher baseline NRP1 and rising ESM1 or claudin-23 combined with declining ESAM or FOXP2 were correctly identified as high-risk trajectories in >80% of converters.

Deployment and clinical utility. Implemented within a laboratory information system, the module normalizes inputs, applies the trained model, and generates diagnostic/prognostic indices with interpretive, color-coded reports and optional integration with imaging or genetics.

Interpretation. Incorporation of NRP1 improves diagnostic precision and early-stage prediction of AD-related neurovascular dysfunction, strengthening the case for endothelial-centric biomarker panels in clinical decision support.

### Example 4—Endothelial Markers in Cognitive Impairment

Cognitively normal participants (CDR 0) with biomarker evidence of AD pathology (i.e., participants with preclinical AD) and healthy controls (n=700; mean age, 67 years; female, 57%; APOE 4+, 34%) were included. Baseline and longitudinal cognitive assessments (CDR, CDR-SB, and PACC) and CSF biomarker measurements (Aβ42/40, p-tau181, tau, NFL, Ng, SNAP-25, VILIP-1, and YKL-40) were obtained in all participants. A subset of participants underwent brain amyloid- (n=537) and tau- (n=287) PET imaging. ROC curves and bootstrapping examined the ability of CSF biomarkers to predict brain amyloid status using amyloid-PET or CSF Aβ42/40. Linear regression and mediation analyses examined the relationship between CSF biomarkers, cognition, amyloid- and tau-PET burden. Linear mixed models examined the ability of CSF biomarkers to predict cognitive and tau-PET burden progression over time.

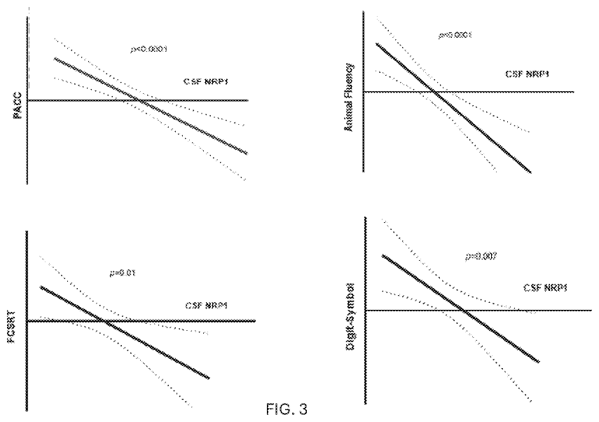

CSF NRP1 levels were significantly elevated in preclinical AD compared to healthy controls. CSF NRP1 predicted brain amyloid status by amyloid-PET or CSF Aβ42/40 (AUC 0.87-0.91, p<0.0001). CSF NRP1 levels were closely associated with amyloid (Aβ42/40 and amyloid-PET), tau (p-tau181 and tau-PET), and neuronal/synaptic injury markers (tau, Ng, SNAP-25, and VILIP-1) (p<0.0001). Higher CSF NRP1 levels were associated with lower PACC (p<0.0001), animal fluency (language) (p<0.0001), FCSRT (episodic memory) (p=0.01), and digit-symbol substitution (executive function) (p=0.007) scores consistent with worse global and domain-specific cognition (FIG. 3). Higher CSF NRP1 levels predicted faster progression in cognitive impairment (p=0.0003) and neocortical tau aggregation (p=0.0001) in Aβ+individuals over time. The relationship between brain endothelial injury and cognition was mediated by neocortical tau aggregation, and direct endothelial-synaptic interactions. Functional pathway interactions showed that NRP1 interacts with pathways involved in synaptic plasticity and hippocampal-mediated learning.

CSF NRP1 levels were higher relative to age-matched controls (p<0.0001), consistent with endothelial activation and BBB dysregulation in AD. In the same cohort, EMS1 (Endocan) was elevated whereas ESAM levels were significantly reduced (p<0.001). This tripartite pattern—NRP 1↑/EMS1↑/ESAM↓—reflects a transition from endothelial quiescence to microvascular activation and junctional destabilization, consistent with BBB disruption and inflammatory endothelial signaling in AD and related dementias.

Across participants, NRP1 co-varied with key AD pathology and neuronal/synaptic injury markers (e.g., p-tau181, total tau, Aβ measures, neurogranin, SNAP-25, VILIP-1, NfL), linking neuropilin-mediated endothelial stress to synaptic injury. Higher baseline NRP1 was associated with faster cognitive decline over follow-up, supporting its value as a prognostic biomarker of neurovascular-driven progression.

### Example 5: Claudin-23 as an Early Indicator of Blood-Brain-Barrier Dysfunction in Preclinical AD

In analyses of cerebrospinal fluid (CSF) from clinically normal individuals with biomarker evidence of early AD, claudin-23 emerged as a sensitive marker of incipient endothelial and tight-junction injury.

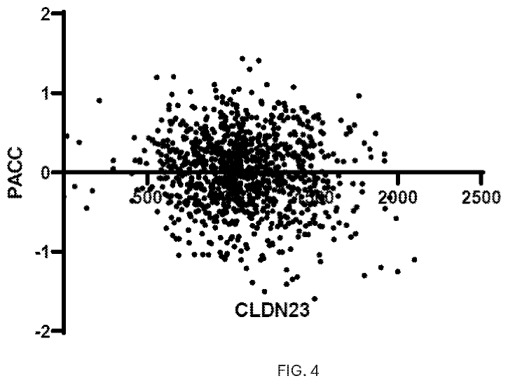

Claudin-23 levels were significantly and inversely correlated with global cognitive performance measured by the Preclinical Alzheimer Cognitive Composite (PACC) (r≈−0.3; p=0.0044), indicating that higher claudin-23 concentrations are associated with worse cognitive function even before overt clinical impairment (FIG. 4).

Because claudins form the paracellular barrier of the blood-brain barrier (BBB), elevated claudin-23 in CSF reflects junctional remodeling and leakage rather than mere expression changes in vascular tissue.

Increased release of claudin-23 fragments or soluble extracellular loops into CSF signifies early disruption of endothelial cohesion, a process that facilitates amyloid deposition, neuroinflammation, and downstream neuronal stress.

The ability of claudin-23 to track subtle cognitive decline in the preclinical stage supports its role as an early-detection biomarker of BBB dysfunction and microvascular injury. When combined with complementary markers—such as NRP1, EMS1 (Endocan), ESAM, FOXP2, and neuroligins—claudin-23 enhances sensitivity for detecting vascular pathology preceding symptomatic AD. Accordingly, inclusion of claudin-23 in diagnostic and monitoring panels enables identification of at-risk individuals before irreversible neurodegeneration, providing a foundation for preventive or disease-modifying therapeutic interventions.

Interpretation (Example 4+Example 5). Together, these findings demonstrate a consistent CSF profile of endothelial activation (NRP1↑, EMS1↑), junctional loss (ESAM↓), and barrier disruption (elevated claudin-23) in cognitive impairment. This coordinated pattern provides a quantitative molecular signature of neurovascular dysfunction that precedes or parallels cognitive decline, establishing its value for diagnosis, risk stratification, and therapeutic monitoring.

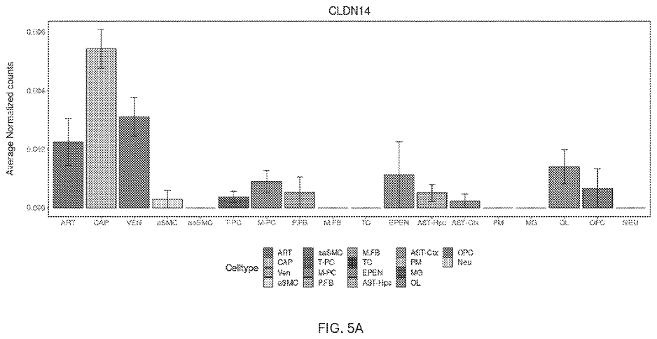

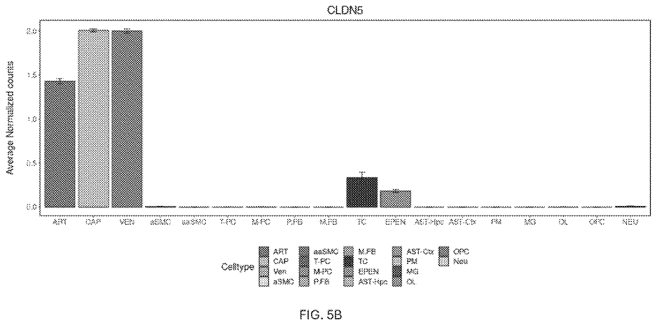

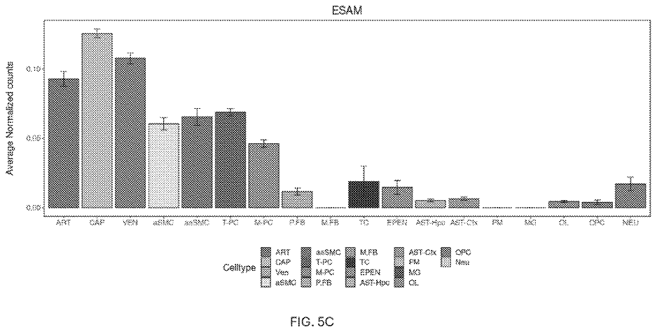

FIG. 5 demonstrates brain endothelial expression patterns of claudin-14, claudin 5, and ESAM supporting their utility as markers of brain endothelial injury.

### DETAILED DESCRIPTION OF THE FIGURES

FIG. 1 shows correlations between cerebrospinal fluid (CSF) NLGN1A levels and established markers of AD pathology. (A) Higher CSF NLGN1, reflective of brain endothelial dysfunction, correlated with lower CSF Aβ42/40 ratios, indicating higher brain amyloid burden. (B) Higher CSF NLGN1, reflective of brain endothelial dysfunction, correlated with higher CSF tau levels, reflecting more severe tau pathology and neurodegeneration.

FIG. 2 shows differences in CSF EMS1 and ESAM levels between cognitively normal controls (CDR 0) and cognitively impaired individuals (CDR>0) with AD. (A) CSF ESM1 was markedly elevated in cognitively impaired participants (p≈2.6×10⁻¹²), and (B) ESAM was significantly reduced (p=0.007) in cognitively impaired participants, compared to cognitively normal controls.

FIG. 3. Higher baseline CSF NRP1 levels were associated with more severe decline in the Preclinical Alzheimer's Cognitive Composite (PACC) scores (global cognition) (p<0.0001), animal fluency (language functions) (p<0.0001), Free and Selective Cued Reminding test (FCSRT; episodic memory) (p=0.01) and Digit-Symbol Substitution test (executive function) (p=0.007) in participants with preclinical AD.

FIG. 4 shows the correlation between CSF claudin-23 levels and Preclinical Alzheimer's Composite (PACC) scores (global cognition). Higher CSF claudin-23 correlated with lower PACC scores (r=−0.27, p=0.0044), reflecting more severe cognitive decline in preclinical AD.

FIG. 5A. Endothelial-enriched expression of claudin-14 (CLDN14) across human brain vascular cell types.

FIG. 5B. Endothelial-restricted expression of claudin-5 (CLDN5) across brain arteriolar, capillary, and venular endothelial populations.

FIG. 5C. Selective expression of endothelial cell-selective adhesion molecule (ESAM) in brain vascular endothelial compartments.

These figures show brain endothelial expression of claudin-14 (A), claudin-5 (B), and ESAM (C) across human brain vascular cell types (arterioles [ART], capillaries [CAP], venules [VEN]) obtained from a human vascular brain atlas (using RNA-seq). Expression Specification was highest in endothelial cells, supporting their use as BBB-specific biomarkers. *ART: Arterial; CAP: Capillary; VEN: Venous; aSMC: Vascular Smooth Muscle Cell; aaSMC: Arteriolar Smooth Muscle Cell; T-PC: Solute transport-Pericyte; M-PC: ECM-regulating Pericyte; P.FB: Perivascular Fibroblast; M.FB: Meningeal Fibroblast; TC: Tcell; EPEN: Ependymal; AST-Hpc: Astrocyte-hippocampus; AST-Ctx: Astrocyte-Cortex; PM: Perivascular Macrophage; MG: Microglia; OL: Oligodendrocyte; OPC: Oligodendrocyte Precursor Cell; NEU: Neuron.

These figures show brain endothelial expression of claudin-14 (A), claudin-5 (B), and ESAM (C) across human brain vascular cell types (arterioles [ART], capillaries [CAP], venules [VEN]) obtained from a human vascular brain atlas (using RNA-seq). Expression was highest in endothelial cells, supporting their use as BBB-specific biomarkers. *ART: Arterial; CAP: Capillary; VEN: Venous; aSMC: Vascular Smooth Muscle Cell; aaSMC: Arteriolar Smooth Muscle Cell; T-PC: Solute transport-Pericyte; M-PC: ECM-regulating Pericyte; P.FB: Perivascular Fibroblast; M.FB: Meningeal Fibroblast; TC: Tcell; EPEN: Ependymal; AST-Hpc: Astrocyte-hippocampus; AST-Ctx: Astrocyte-Cortex; PM: Perivascular Macrophage; MG: Microglia; OL: Oligodendrocyte; OPC: Oligodendrocyte Precursor Cell; NEU: Neuron.

It is to be understood that the above description is intended to be illustrative and not restrictive. For example, the above-described embodiments (and/or aspects thereof) may be used in combination with each other. In addition, many modifications may be made to adapt a particular situation, method, system device, or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

This written description uses examples to disclose the various embodiments of the invention, including the best mode, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements or steps that do not differ from the literal language of the claims, or if the examples include equivalent structural elements or steps with insubstantial differences from the literal language of the claim.

1. A method for diagnosing a neurological disease or disorder in a patient in need thereof, comprising:
(a) obtaining a biological sample from the patient;
(b) measuring levels of at least one biomarker;
(c) comparing biomarker levels to a control or reference standard; and
(d) diagnosing the patient as having the neurological disease or disorder when at least one biomarker level is altered relative to the control or reference standard.

2. The method of claim 1, wherein the biological sample comprises a sample selected from a group consisting of cerebrospinal fluid, plasma, serum, blood, urine, saliva, and any combination thereof.

3. The method of claim 1, wherein the neurological disease or disorder is selected from a group consisting of Alzheimer's disease, vascular dementia, frontotemporal dementia, Lewy body dementia, and any combination thereof, and wherein alterations in at the least one biomarker selected from a group consisting of endothelial cell-selective adhesion molecule (ESAM), endothelial cell-selective molecule-1 (ESM1 or Endocan), neuropilin-1 (NRP1), neuropilin-2 (NRP2), endothelins (ET-1, ET-2, and ET-3), claudin-14 (CLDN14), claudin-23 (CLDN23), forkhead box protein P2 (FOXP2), zinc finger of the cerebellum 1 (ZIC1), neuroligins 1-4, and any combination thereof, are indicative of blood-brain barrier disruption, endothelial dysfunction, and endothelial contributions to neurodegeneration associated with the neurological disease or disorder.

4. The method of claim 1, wherein the neurological disease or disorder is selected from a group consisting of Alzheimer's disease, vascular dementia, frontotemporal dementia, Lewy body dementia, and any combination thereof; the diagnosing is based on stage-dependent alterations in neuroligin and endothelial biomarker levels, wherein
(i) increased neuroligin levels together with elevated expression of at least one endothelial biomarker selected from a group consisting of endothelial cell-selective molecule-1 (ESM1 or Endocan), neuropilin-1 (NRP1), neuropilin-2 (NRP2), endothelins (ET-1, ET-2, or ET-3), claudin-14 (CLDN14), claudin-23 (CLDN23), and any combination thereof, and decreased ESAM level are indicative of early or preclinical disease; and
(ii) decreased neuroligin levels together with decreased FOXP2, decreased ZIC1, decreased ESAM, with a combination of persistent elevation of endothelial injury markers are indicative of symptomatic or advanced disease.

5. The method of claim 1, wherein the neurological or neurovascular disorder is selected from a group consisting of traumatic brain injury, spinal-cord injury, ischemic or hemorrhagic stroke, post-ischemic encephalopathy, subarachnoid hemorrhage, cerebral hypoxia, and any combination thereof, and wherein alterations in at least one biomarkers selected from a group consisting of ESM1, endothelins 1-3, claudin-14, claudin-23, NRP1, NRP2, ESAM, FOXP2, ZIC1, and any combination thereof are indicative of blood-brain barrier disruption, vascular leakage, endothelial activation, or reperfusion injury.

6. The method of claim 1, wherein the neurological disease or disorder is an autoimmune or inflammatory demyelinating disease selected from a group consisting of multiple sclerosis (MS), acute disseminated encephalomyelitis (ADEM), neuromyelitis optica spectrum disorder (NMOSD), MOG-antibody disease (MOGAD), and any combination thereof, and wherein alterations in at least one biomarker is selected from a group consisting of claudins 1-34 (including claudin-14 and claudin-23), EMS1 (Endocan), ESAM, endothelins 1-3, neuroligins 1-4 (and subtypes thereof), NRP1, NRP2, FOXP2, ZIC1, and any combination thereof are indicative of blood-brain barrier dysfunction, endothelial activation, neuroinflammatory lesion activity, and relapse risk.

7. The method of claim 1, wherein the neurological disease or disorder is selected from the group consisting of central nervous system vasculitis, infectious meningoencephalitis, neuroinflammatory conditions, and any combination thereof, and alterations in the at least one biomarker comprising ESM1, endothelin, claudins, NRP1, NRP2, ESAM, FOXP2, and ZIC1 are indicative of endothelial activation and vascular compromise.

8. The method of claim 1, wherein biomarker data obtained from at least one biological samples are used to train, validate, or update a machine-learning and artificial-intelligence model configured for automated classification or stratification of neurological or neurovascular disease subtypes, and wherein feature-importance weighting and model attribution identifies one or more biomarkers selected from a group consisting of ESM1 (Endocan), endothelins 1-3, claudins 1-34 (including claudin-14 and claudin-23), ESAM, NRP1, NRP2, FOXP2, ZIC1, neuroligins 1-4, neuroligin subtypes 1A, 1B, 1C, 2C, 2ECD, 3A, 3B, 4X, and 4Y, and any combination thereof as principal contributors to vascular-risk prediction, disease-stage classification, and therapeutic-response modeling.

9. The method of claim 1, wherein at least one biomarker is selected from a group consisting of claudins 1-34, ZIC1, FOXP2, neuroligins 1-4 and subtypes 1A, 1B, 1C, 2C, 2ECD, 3A, 3B, 4X, 4Y, endothelins 1-3, ESAM, ESM1, neuropilins (NRP1 and NRP2), and any combination thereof.

10. The method of claim 9, wherein the at least one biomarker comprises at least one neuroligin and at least one endothelial biomarker selected from a group consisting of ESAM, ESM1 (Endocan), claudin-14, claudin-23, an endothelin (ET-1, ET-2, ET-3), a neuropilin (NRP1 or NRP2), and any combination thereof.

11. The method of claim 10, wherein altered neuroligin levels together with increased biomarker selected from a group consisting of ESM1, claudin-14, claudin-23 levels, and combination thereof together with decreased ESAM levels and increased NRP1, NRP2, and endothelin levels relative to control are indicative of Alzheimer's disease.

12. The method of claim 10, wherein a biomarker panel comprising neuroligin-1 and endothelin-1 levels detects early Alzheimer's disease.

13. The method of claim 10, wherein decreased levels or dysregulated FOXP2 and ZIC1 levels in combination with altered neuroligin and endothelial biomarker levels are diagnostic of Alzheimer's disease,
wherein transient increases in FOXP2 or ZIC1 may occur in acute vascular stress, and sustained decreases indicate chronic neurovascular injury.

14. The method of claim 10, wherein stage-dependent alterations in neuroligin and endothelial biomarker levels are evaluated longitudinally in the at least one biological sample to generate a trajectory-based composite index distinguishing preclinical from symptomatic Alzheimer's disease.

15. A method for monitoring progression of a neurological disease or disorder in a patient, comprising:
(a) obtaining biological samples from the patient at least two or more timepoints;
(b) measuring levels of at least one biomarker;
(c) comparing biomarker levels between timepoints; and
(d) determining progression, stabilization, or improvement based on longitudinal changes in the said biomarker levels.

16. The method of claim 15, wherein serial increases in the levels of at least one biomarker selected from a group consisting of NRP1 or NRP2, endothelin, ESM1, claudin-14, claudin-23, and any combination thereof, together with decreased in levels of at least one biomarker selected from a group consisting of neuroligins, ESAM, FOXP2, ZIC1, and any combination thereof relative to baseline predict progression from preclinical to symptomatic Alzheimer's disease.

17. The method of claim 15, wherein longitudinal increases in levels of claudin-14, claudin-23, ESM1, NRP1, and endothelin correlate with worsening outcome after stroke or traumatic brain injury.

18. The method of claim 15, wherein the at least one biomarker is selected from a group consisting of claudins 1-34, ZIC 1, FOXP 2, neuroligins 1-4 (including subtypes 1A, 1B, 1C, 2C, 2ECD, 3A, 3B, 4X, 4Y), endothelins 1-3, ESAM, ESM1, neuropilins (NRP1 and NRP2), and combination thereof.

19. The method of claim 18, wherein the neurological disease or disorder patient carries an apolipoprotein E4 (APOE4) allele, and wherein progression in the at least one biomarker is monitored in the said patient carrying the apolipoprotein E4 (APOE4) allele.

20. The method of claim 18, wherein progression is determined using a composite index computed from neuroligin and endothelial biomarker levels that reflects worsening neurovascular dysfunction relative to baseline.

21. A method of monitoring effectiveness or safety of a therapy for a neurological disease or disorder in a patient, comprising:
(a) obtaining a baseline biological sample from the patient before or at initiation of therapy;
(b) measuring levels of at least one biomarker;
(c) obtaining a subsequent biological sample during or after therapy
(d) comparing biomarker levels between a baseline and subsequent sample; and
(e) determining whether to continue, modify, or discontinue therapy based on the said comparison.

22. The method of claim 21, wherein biomarker monitoring is used to determine whether to reinitiate or permanently discontinue anti-amyloid therapy.

23. The method of claim 21, wherein the therapy comprises administration of an anti-amyloid monoclonal antibody, including lecanemab, donanemab, and a functionally equivalent disease-modifying agent for dementia, and wherein biomarker level changes are used to monitor risk of amyloid-related imaging abnormalities (ARIA).

24. The method of claim 23, wherein (a) a chronic biomarker pattern characterized by decreased levels of ESAM, FOXP2, and ZIC1 levels together with persistently elevated ESM1, claudin-14, claudin-23, endothelin, NRP1, and NRP2 levels indicates increased susceptibility to amyloid-related imaging abnormalities (ARIA), and (b) an acute biomarker pattern characterized by further increases in ESM1, claudin-14, claudin-23, endothelin, NRP1, and NRP2 and transient elevation or fluctuation of ESAM, FOXP2, or ZIC1 relative to baseline indicates active or imminent ARIA development.

25. The method of claim 23, wherein amyloid-related imaging abnormality (ARIA) resolution or recovery is indicated by (a) decreased levels of ESM1, endothelin, claudin-14, claudin-23, NRP1, and NRP2 relative to values observed during ARIA onset, and (b) stabilization or restoration of ESAM, FOXP2, and ZIC1 levels toward baseline following therapy modification or interruption.

26. A kit for diagnosing or monitoring a neurological disease or disorder, comprising:
(a) a panel of binding reagents selected from a group consisting of antibodies, antibody fragments, aptamers, nucleic-acid probes, and other binding reagents specific for at least one biomarker, and any combination thereof;
(b) calibration standards; and
(c) instructions for comparing biomarker levels to a control or reference to determine disease presence, stage, progression, or therapy response.

27. The kit of claim 26, wherein the panel of binding reagents is provided within a multiplex immunoassay, microarray, nucleic-acid detection platform, and other analytical system configured for simultaneous quantification of the said biomarkers, the kit further configured for use in enzyme-linked immunosorbent assay (ELISA), proximity-extension assay, mass-spectrometry-based quantification, and equivalent analytical formats.

28. The kit of claim 26, wherein the calibration standards comprise at least one component selected from a group consisting of pre-quantified recombinant and synthetic peptides, protein fragments, full-length proteins, nucleic-acid constructs, and any combination thereof, each corresponding to at least one biomarker selected from a group consisting of claudins 1-34 (including claudin-14 and claudin- 23), ESAM, ESM1 (Endocan), FOXP2, ZIC1, neuroligins 1-4, neuropilins (NRP1 and NRP2), endothelins 1-3, and any combination thereof.

29. The kit of claim 26, wherein the at least one biomarker is selected from a group consisting of claudins 1-34, ZIC 1, FOXP 2, neuroligins 1-4 and subtypes 1A, 1B, 1C, 2C, 2ECD, 3A, 3B, 4X, 4Y, endothelins 1-3, ESAM, ESM1, neuropilins (NRP1 and NRP2), and any combination thereof, and wherein the kit comprises capture reagents for at least one neuroligin and at least one endothelial biomarker selected from a group consisting of ESAM, ESM1, claudin-14, claudin-23, an endothelin, a neuropilin (NRP1 or NRP2), and any combination thereof.

30. A computer-implemented method for generating a disease risk or stage score in a patient, comprising:
(a) receiving biomarker level data measured from a biological sample;
(b) normalizing at least one biomarker level against a reference database or baseline;
(c) applying a classifier trained to distinguish between neurological disease states and control subjects; and
(d) outputting a diagnostic or prognostic score indicating a likelihood or stage of disease;
wherein the classifier is configured to assign feature weights comprising (a) increasing a computed probability of early Alzheimer's disease for patterns characterized by elevated levels of neuroligins, ESM1, endothelin, claudin-14, claudin-23, NRP1, and NRP2, together with reduced levels of ESAM, FOXP2, and ZIC1; and
(b) increase the computed probability of advanced Alzheimer's disease for patterns characterized by reduced levels of neuroligins, FOXP2, and ZIC1.

31. The method of claim 30, further comprising a non-transitory computer-readable medium storing software executable instructions configured to (a) receiving the at least one biomarker-level data from at least one assay, (b) normalizing levels of at least one biomarker relative to at least one reference datasets, and (c) generating diagnostic, prognostic, and therapeutic-response indices, and wherein the further including stage-specific classification of early (neuroligin increase ) versus late (neuroligin decreased) disease.

32. The method of claim 31, further comprising integration of said machine-learning or artificial-intelligence model with a clinical-decision support platform configured for real-time disease stratification, risk prediction, or therapy-response optimization.
